(19) United States
(12) Patent Application Publication
(10) Pub. No.: US 2008/0193540 A1
(43) Pub. Date: Aug. 14, 2008

(54) Solid, Oral Drug Form Which Has Been Designed to Prevent Misuse

(75) Inventors: Gerard Soula, Meyzieu (FR); Frederic Dargelas, Pessac (FR)

Correspondence Address: PATTON BOGGS LLP, 8484 WESTPARK DRIVE, SUITE 900, MCLEAN, VA, 22102, US

(73) Assignee: Flamel Technologies, Venissieux (FR)

(21) Appl. No.: 11/791,336

(22) PCT Filed: Nov. 21, 2005
(86) PCT No.: PCT/FR2005/050969

§ 371 Date: Jan. 9, 2008

(30) Foreign Application Priority Data
Nov. 23, 2004 (FR) 0412428

(51) Int. Cl. A61K 9/14; A61K 47/44; A61K 47/14; A61K 47/32; A61K 47/36; A61K 47/04; A61P 43/00

(52) U.S. Cl. 424/489; 514/785; 514/786; 514/772.4; 514/772.5; 514/779; 514/781; 514/770

(57) ABSTRACT

The invention relates to the field of solid medicaments that are intended for the oral administration of active ingredients. The aim of the invention is to prevent the improper use of solid, oral medicaments for any user other than the therapeutic use(s) officially approved by the appropriate public health authorities. More specifically, the invention relates to a solid, oral drug form which is characturised in that it comprises: A) at least one caking agent; and B) at least one viscosifying agent, such as to prevent the misuse thereof.

### FIELD OF THE INVENTION

The present invention relates to the field of solid medicaments that are intended for the oral administration of active ingredients.

The active ingredients (AIs) considered are pharmaceutical AIs, and in particular those classified in the category of narcotic products. The latter are those which, when abused, can lead to drug addiction-related behavior.

For the purpose of the present disclosure, the expression “AI” denotes both a single active ingredient and a mixture of several active ingredients.

The aim of the present invention is to prevent the improper misuse of solid oral medicaments for any use other than the therapeutic use(s) officially approved by the appropriate public health authorities. In other words, it is a question of preventing intentional or unintentional misuse of solid oral medicaments.

Situation of the Problem

Misuse is mainly encountered in the following cases:
a. addictive behavior (drug addiction, doping),
b. criminal behavior (chemical dependency),
c. use of a medicament in a manner that does not comply with the medical recommendations (posology), inadvertently or due to disabilities affecting the patient,
d. self-medication.

In case a. (or even in case b.), individuals who have the intention of misusing the solid oral medicament will generally apply themselves to making it either into a pulverulent form which can be inhaled, or into a liquid form which can be injected using a syringe.

The solid/powder to be snorted conversion is carried out by crushing.

The obtaining of an injectable liquid form from a solid oral medicament involves a step consisting of aqueous or alcoholic extraction of the targeted AI. This extraction can be preceded by crushing.

Methods of administration by inhalation or by injection are particularly suitable for drug addicts because they are methods which make it possible to accentuate the effects of the AI and which promote its absorption in the body over short periods of time. When this powder is aspirated via the nose or dissolved in water and injected, the desired doping or euphoria-inducing effects of the AI manifest themselves very rapidly and in an exacerbated manner.

The misuse of solid oral medicaments can also be observed when the medicament is chewed before being swallowed, instead of being swallowed rapidly in accordance with the posology. This is particularly observed in the case of sustained-release drug forms where the dose of active ingredient per pharmaceutical presentation form (tablet, gelatin capsule) can be relatively high compared with conventional immediate-release products. In fact, a medicament for sustained release of AI contains a higher dose of AI since it must cover the needs over a longer period (for example: administration of the same amount of active ingredient in a single dose per day instead of several. The fact of biting or crushing the medicament and swallowing it no longer guarantees the initial controlled release and thus allows a massive administration of AI, causing the desired doping or euphoria-inducing effects.

The risks associated with addictive behavior (a.) and criminal behavior (b.) and with self-medication (d.) are obvious. It will be recalled that the misuse of medicaments by injection is a serious situation: the excipients may be responsible for local tissue necroses, for infections, and for respiratory and cardiac problems.

As regards abuses (c.) of the use of a medicament that are related to inattention and/or to disabilities of the patient, they can also have serious consequences. For example, the chewing, before swallowing, of forms for modified release of AI converts the medicament into an immediate-release form. Thus, at best, the medicament is ineffective after a very short period of time, and, at worst, it becomes toxic.

There clearly exists therefore a serious public health problem related to the misuse of medicaments, and in particular of solid oral medicaments.

This increasing phenomenon is becoming more and more worrying to health authorities, which are multiplying appeals for the development of drug forms for preventing improper use.

### PRIOR ART

To the applicant's knowledge, the only attempts to tackle this problem have consisted in adding, to the medicaments concerned, compounds that are chemically active against misuse.

This solution has certain dangers for users, including for use under the approved conditions. In addition, combinations of AI and of other active compounds are tricky to control and are received with suspicion by public health authorities responsible for granting marketing authorizations.

US-A-2003/0068371 describes an oral pharmaceutical formulation comprising an opiate AI, an antagonist of this AI and a gelling agent (e.g. xanthan gum). The gelling agent is presented as conferring on the formulation a viscosity such that it cannot be administered nasally or parenterally. The presence of this antagonist is a major disadvantage with regard to the medical risks possibly run by users. In addition, this pharmaceutical form can be made into pulverulent form and, consequently, can be the subject of misuse by nasal administration.

### OBJECTIVES OF THE INVENTION

Under these circumstances, one of the essential objectives of the present invention is to overcome the shortcomings of the prior art.

Another essential objective of the invention is to provide novel solid oral medicaments, the misuse of which will be made very difficult or even impossible, in particular for the abovementioned cases (a.)(b.)(c.)(d.), without resorting to substances, other than the AI, that may be pharmaceutically active and therefore dangerous for users.

Another essential objective of the invention is to provide a novel solid oral medicament that makes it possible to prevent the fraudulent misuse of the properties of the AI that it contains, by preventing any conversion of the medicament that means it can be taken orally, nasally and/or by injection (intravenous, subcutaneous, intramuscular, etc.) outside the therapeutic context. In so doing, the risks associated with these derivatives will be prevented or at the very least greatly reduced.

Another essential objective of the invention is to provide a novel solid oral medicament that makes it possible to prevent misuse, while at the same time guaranteeing, for the patient normally followed-up, a quality of treatment, in particular a dose, in accordance with his or her needs.

Another essential objective of the invention is to provide a novel solid oral medicament that makes it possible to prevent misuse without affecting the pharmacological properties of the medicament, and without causing the patient, who uses the medicament normally, to run any additional risks and, finally, without being detrimental to the comfort of the latter during administration.

Another essential objective of the invention is to provide a novel solid oral medicament that makes it possible to prevent misuse, and that is simple to obtain and does not cause its cost price to increase.

### SUCCINCT DESCRIPTION OF THE INVENTION

In order to attain these objectives, it is to the inventors, credit to have reformulated the problem to be solved, by no longer stating it in chemical terms, but rather in physicochemical terms. This new approach has allowed them to discover, surprisingly and unexpectedly, that it is advisable to involve, in the medicament, the misuse of which it is sought to prevent, a combination of agents which have a physicochemical method of action and the purpose of which is to impede, or even make impossible, any intentional or unintentional act of misuse.

Thus, the invention relates, mainly, to a solid oral drug form, characterized in that it comprises:
A) at least one caking agent,
and
B) at least one viscosifying agent; so as to as prevent misuse.

The drug form according to the invention solves the stated problem and meets the objectives set, effectively, simply and economically, using physicochemical means. The latter are completely harmless for anyone using the drug normally. They are pharmacologically neutral (inert) compounds approved by the pharmacopeia and the public health authorities responsible for granting marketing authorizations for medicaments.

The caking agent A) makes it difficult to crush the solid bulk drug form and does not make it possible to obtain a pulverulent form suitable for administration by nasal aspiration.

The viscosifying agent B) makes it difficult to extract the AI from the drug form, thus preventing misuse. Moreover, B) makes it difficult, or even impossible, to inject it parenterally.

### DETAILED DESCRIPTION OF THE INVENTION

The present invention relates to all the unit or divided, immediate-release or sustained release, solid oral drug forms which prevent misuse of the medicament, in particular of the AI that it contains, both by injection (parenteral) and by nasal or oral administration.

These forms can, for example, be tablets or gelatin capsules.

As an addition to, or in place of, the combination of agents A) and B), the invention can be characterized by at least two other essential characteristics, detailed below.

Preferably, all or part of the AI of the drug form according to the invention is contained in microparticles.

As another obstacle to misuse, possibly as an addition to agents A) and B), the drug form according to the invention comprises inert insoluble beads which have an average diameter of greater than or equal to 1.25 times, preferably 1.5 times, and even more preferably twice, the average diameter of the microparticles of AI.

These insoluble beads, i.e. insoluble in an aqueous or aqueous-alcoholic medium for the purpose of the invention, cannot be compressed. The crushing stresses will therefore be mainly borne by the beads due to the fact that they are larger than the microparticles containing the AI. Thus, the microparticles containing the AI will be preserved against the crushing.

According to a preferred characteristic of the invention, the caking agent A) is chosen from those capable of ensuring, in the event of crushing of the drug form, that the latter is converted into a non-pulverulent product.

In fact, as indicated above, misuse by nasal inhalation assumes that the user will crush the solid oral form in order to convert it into a powder to be snorted.

In addition, a perpetrator of misuse may also seek to extract the AI using an aqueous and/or alcoholic solution, in order to concentrate it.

With these observations as a starting point, the inventors reasoned, inventively, that it was advisable to complicate (or even prevent) these processes of crushing (or any other mechanical treatment for converting a solid into powder) and of extraction by using at least one caking agent having the function of converting the drug form into a nonpulverulent product, for example into a viscous paste that cannot be manipulated, as soon as it is taken out of its bulk solid state.

The drug form thus contains at least one hydrophobic agent A) (wax, oil). If the medicament improperly used is crushed (in order to be snorted as a powder), the hydrophobic compound plays the role of a dry binder. The AI and the various excipients form a mixture which cannot be finely divided (heavy pasty powder) and prevent its aspiration via the nose.

Similarly, it is well known that the extraction of a compound to be concentrated from a viscous paste is extremely difficult.

Even more preferably, the caking agent A) is chosen from the class of hydrophobic compounds that act as a dry binder, preferably:
from the group comprising cottonseed oils, soybean oils, palm oils, castor oils and mixtures of all or some of these oils; and/or
from the group of waxes, and even more preferably from the subgroup of waxes comprising hydrogenated cottonseed oils, hydrogenated soybean oils, hydrogenated palm oils, glyceryl behenates, hydrogenated castor oils, tristearins, tripalmitins, trimyristins, yellow waxes, hard fats, anhydrous dairy fats, lanolins, glyceryl palmitostearates, glyceryl stearates, lauric acid macrogolglycerides, cetyl alcohols, polyglyceryl diisostearates, diethylene glycol monostearates, ethylene monostearates, omegas 3 and mixtures of all or some of these waxes; and/or
from the group of fatty bases for suppositories comprising glycerol, triglycerides, theobroma oils, cacao butters and mixtures of all or some of these products.

As regards the amount of caking agent A) that can be introduced into the drug form according to the invention, a concentration ranging from 1% to 90% weight/weight relative to its total mass of the drug form is, for example, anticipated.

It is therefore a notable characteristic of the drug form according to the invention that it cannot be converted into a dry form that can be administered by nasal aspiration.

Preferably, the viscosifying agent B) is chosen from those capable of rendering noninjectable the AI contained in the drug form.

The use of this viscosifying agent B) is more especially (but not in a limiting manner) related to misuse by parenteral injection of the AI and of the excipients of a drug form.

In fact, the perpetrator of such a misuse must convert a solid product into an injectable liquid that is as concentrated as possible in terms of narcotic AI. As explained above, this involves an aqueous and/or alcoholic extraction. The misuse is continued by filling a syringe with the liquid obtained, before injection.

In this context, the inventors' original idea was to provide a viscosifying agent B) which, as soon as it is brought into contact with a liquid, causes an increase in viscosity, making injection using a syringe impossible. This high viscosity prevents both filling and emptying of the syringe.

The drug form of the invention contains at least one agent B) advantageously chosen from viscosifying polymers. When the drug form is mixed with a solvent (aqueous or organic), the polymer B) participates in increasing the viscosity of and/or in gelling the medium, firstly limiting the dissolution of the AI and secondly preventing the possibility of taking up or injecting the solution by means of a syringe.

It appears that, as regards the viscosifying agent B) (like the caking agent A), the mechanism that provides an obstacle to misuse by injection is purely physicochemical and, consequently, neutral with respect to anyone using the medicament normally.

Preferably, the viscosifying agent B) is chosen from the following groups of polymers:
polyacrylic acids, and/or
polyalkylene glycols (e.g. polyethylene glycol), and/or
polyvinylpyrrolidones, and/or
gelatins, and/or
pectins, and/or
polysaccharides, preferably from the subgroup comprising: sodium alginate, pectins, guars, xanthans, carrageenans, gellans and cellulose derivatives (e.g. hydroxypropylmethylcellulose, methylcellulose, hydroxyethylcellulose, carboxymethylcellulose), and mixtures thereof.

As regards the amount of viscosifying agent B) that may be introduced into the drug form according to the invention, a concentration ranging from 1% to 90% weight/weight relative to the total mass of the drug form is, for example, anticipated.

It is therefore a notable characteristic of the drug form according to the invention that it cannot be converted into an injectable form.

Advantageously, the drug form combines agents A) and B), described above, for preventing misuse in a manner suitable for any medicament and/or any AI.

In summary, the use of A) and of B), in the drug form according to the invention, makes it nonsnortable and noninjectable.

It is not possible to convert it either into a volatile powder or into a liquid concentrated in AI that can be drawn up and can be expelled using a syringe.

The multimicroparticulate nature of the drug form and/or the presence of inert insoluble beads contribute(s) to the abovementioned result.

The drug form according to the invention may comprise immediate-release AI and/or modified-release AI.

Preferably, the neutral insoluble beads are chosen from the group of following substances: celluloses and insoluble derivatives thereof, polymethacrylic resins and derivatives thereof, silicas, talc, semolina, bentonite and mixtures thereof.

As specified above, forms for modified release of AI contain higher doses of AI than immediate-release forms. This has generated, in particular on the part of drug addicts, misuse consisting in crushing and/or chewing the forms for modified release of AI, so as to destroy the barriers provided in order to ensure modified release of the AI and thus to gain access to higher concentrations of narcotic AI.

It is advisable to distinguish between, firstly, chewing and, secondly, crushing.

As regards chewing, it should be underlined that it can be intentional or unintentional. In fact, certain patients suffering from disabilities—e.g. Parkinson's disease—are not able to comply with the posology which prescribes swallowing without biting down.

In order to remedy this, the invention proposes a multimicroparticulate form in which the microparticles of AI have a small average diameter in order to escape mastication.

As regards crushing, it constitutes a necessary and essential step during improper use of the medicament, regardless of the misuse, prior to extraction of the AI.

Now, the characteristics specific to. the invention, namely combination of A) and B), AI in multimicroparticulate form and presence of insoluble inert beads having a diameter larger than the microparticles of AI, protect the latter against any effective form of crushing. It is thus impossible to release the AI out of the-microcapsule or to extract it.

Thus, the present invention proposes a first specific embodiment for effectively combating misuse by chewing mentioned in the paragraphs above.

In accordance with this first specific embodiment, all or part of the AI of the drug form according to the invention is contained in microparticles.

The present invention also proposes a second specific embodiment for effectively combating misuse by crushing mentioned in the above paragraphs.

In accordance with this second specific embodiment,
all or part of the AI of the drug form according to the invention is contained in microparticles,
the drug form comprises at least one caking agent A) and at least one vi.scosifying agent B),
the drug form comprises insoluble inert beads as defined above.

The “multimicroparticulate” form has the advantage of providing a dose of AI dispersed in a plurality of microparticles, such that access to the AI deprived of its modified-release barriers, by crushing and/or chewing, is significantly restricted. In fact, a certain number of the microparticles escape the destruction of the means of modified release of the AI, due to their very small size.

In the case of a sugar-coated matrix tablet, it, is sufficient to bite down on the tablet in order to reveal the core containing the AI, and then to swallow it. In the case of a divided form composed of numerous microparticles, it is necessary to individually fracture the coating of a large number of spherical objects, the size of which, (of the order of magnitude of the gaps between the teeth) is such that, firstly, it is difficult to bite down on them and that, secondly, the natural phenomenon of salivation and swallowing means that the microparticles have a natural tendency not to remain in the mouth and to escape chewing. This natural phenomenon may be advantageously amplified by the addition of excipients such as sugars, acidifying agents and sapidity agents, for example.

A denotable characteristic of the drug form according to the invention is that the extraction of the AI by chewing and/or crushing is not effective.

Once again, this microparticulate dispersity advocated by the invention is physicochemical in nature and cannot therefore have any harmful effects on normal users.

According to a notable arrangement of the invention, at least part of the microparticles of the drug form are microcapsules for modified release of AI.

These microcapsules advantageously each consist of a core comprising AI and of a single layer or multilayer coating surrounding the core and controlling the modified release of the AI.

Preferably, the “multimicroparticulate” drug form according to the invention is characterized in that the microparticles and/or the microcapsules have an average diameter of less than or equal to 1000 μm, preferably less than or equal to 500 μm, and more preferably a diameter of less than or equal to 300 μm.

The misuse is characterized practically most of the time by a need to bite down on the medicament and swallow it, or else to crush it more finely in order to inject oneself with it or to snort it.

Judiciously, the preferred drug form will therefore be in divided form, the narcotic active ingredient being contained in a very large number of microparticles of less than 500 μm, and preferably less than 300 μm, in size. In this form, the crushing of small spherical objects is more difficult than a simple sugar-coated matrix tablet, and it becomes virtually impossible to bite down on them.

By way of nonlimiting examples, it may be indicated that the present invention:
applies to AIs belonging to at least one of the following families of active substances: amphetamines, analgesics, appetite suppressants, antidepressants, antiepileptics, antimigraine agents, antiparkinsonian agents, antitussives, anxiolytics, barbiturates, benzodiazepines, hypnotics, laxatives, neuroleptics, opiates, psychostimulants, psychotropic agents, sedatives, stimulants;
applies to the compounds chosen from the following compounds: methylphenidate, Fentanyl, Alfentanyl, Pentazocine, Pethidine, Phenoperidine, Remifentanil, Sufentanil, Acetorphine, Acetylalphamethylfentanyl, Acetylmethadol, Alfentanil, Allylprodine, Alphacetylmethadol, Alphameprodine, Alphamethadol, Alphamethylfentanyl, Alpha-methylthiofentanyl, Alphaprodine, Anileridine, atropine, Benzethidine, Benzylmorphine, Beta-hydroxyfentanyl, Beta-hydroxymethyl-3-fentanyl, Betacetylmethadol, Betameprodine, Betamethadol, Betaprodine, Bezitramide, buprenorphine, Dioxaphentyl butyrate, Cannabis, Ketobemidone, Clonitazene, codeine, Coca, Cocaine, Codoxime, Concentrate of poppy straw, Desomorphine, Dextromoramide, Diampromide, Diethylthiambutene, Difenoxine, Dihydroetorphine, Dihydromorphine, Dimenoxadol, Dimepheptanol, Dimethylthiambutene, Diphenoxylate, Dipipanone, Drotebanol, Ecgonine, ephedrine, Ethylmethylthiambutene, Etonitazene, Etorphine, Etoxeridine, Fentanyl, Furethidine, Heroin, Hydrocodone, Hydromorphinol, Hydromorphone, Hydroxypethidine, Isomethadone, Levomethorphane, Levomoramide, Levophenacylmorphane, Levorphanol, meperidine, Metazocine, Methadone, Methyldesorphine, Methyldihydromorphine, Methyl-3-thiofentanyl, Methyl-3-fentanyl, Metopon, Moramide, Morpheridine, morphine, MPPP, Myrophine, Nicomorphine, Noracymethadol, Norlevorphanol, Normethadone, Normorphine, Norpipanone, Opium, Oxycodone, Oxymorphone, Para-fluorofentanyl, PEPAP, Pethidine, Phenampromide, Phenazocine, Phenomorphane, Phenoperidine, Piminodine, Piritramide, Proheptazine, propanolol, Properidine, Racemethorphane, Racemoramide, Racemorphane, Remifentanil, Sufentanil, Thebacone, Thebaine, Thiofentanyl, Tilidine, Trimeperidine, Acetyldihydrocodeine, Codeine, Dextropropoxyphene, Dihydrocodeine, Ethylmorphine, Nicocodine, Nicodicodine, Norcodeine, Pholcodine, Propiram,
   and mixtures thereof.

### EXAMPLES

The following examples are given by way of illustration of the present invention. They in no way constitute a limit to the possibilities.

### Example 1

In this example, a high viscosity of the solution resulting from the dissolution of the excipients of an improperly used medicament is targeted. 450 g of microparticles of placebos consisting of neutral sugar cores, of between 200 and 300 μm in diameter, are film-coated with a solution S1 containing 33.75 g of polyvinylpyrrolidone (Kollidon 90 F), 78.75 g of sodium alginate and 1012.5 g of ethanol. 50 g of these microparticles are then mixed with 50 g of a low-melting-point wax (Gelucire 44/14). 250 mg of these microparticles are mixed with 1 ml of water adjusted to 0.1M CaCl₂ and neutral pH. The resulting solution is too viscous to be injected. The crushed form is pasty (nonpulverulent).

### Example 2

50 g of the microparticles of example 1 are mixed with 50 g of a low-melting-point wax (Gelucire 44/14). 50 g of cellulose spheres of between 450 and 550 μm in diameter are added to this preparation. Crushing this preparation with a mortar produces a nonpulverulent paste. The microscopic observation shows that the sugar particles have withstood the crushing.

### Example 3

In this example, protection against fraudulent use of the medicament by the nose and via injection is targeted. For this, a mixture consisting of 100 mg of crosslinked polyacrylic acid (Carbopol® 934P), 160 mg of sodium diclofenac (as model active ingredient), 100 mg of hydrogenated plant oil wax (Lubritab®), 10 mg of magnesium stearate and 130 mg of lactose is tableted.

Dry crushing of these constituents produces a nonpulverulent waxy paste that prevents aspiration thereof by the nose. Solubilization of this tablet results in a solution that is too viscous to be injected.

What is claimed is:

1. A solid oral drug form, characterized in that it comprises:
C) at least one caking agent,
and
D) at least one viscosifying agent; so as to prevent misuse.

2. The drug form as claimed in claim 1, characterized in that all or part of the AI of this drug form is contained in microparticles.

3. The drug form in particular as claimed in claim 1 or 2, characterized in that it comprises inert insoluble beads which have an average diameter of greater than or equal to 1.25 times, preferably 1.5 times, and even more preferably twice, the average diameter of the microparticles of AI.

4. The drug form as claimed in one of the preceding claims, characterized in that the caking agent A) is chosen from those capable of ensuring, in the event of crushing of the drug form, that the latter is converted into a nonpulverulent product.

5. The drug form as claimed in claim 1 or 4, characterized in that the caking agent A) is chosen from the class of hydrophobic compounds that act as a dry binder, preferably:
from the group comprising cottonseed oils, soybean oils, palm oils, castor oils and mixtures of all or some of these oils; and/or
from the group of waxes, and even more preferably from the subgroup of waxes comprising hydrogenated cottonseed oils, hydrogenated soybean oils, hydrogenated palm oils, glyceryl behenates, hydrogenated castor oils, tristearins, tripalmitins, trimyristins, yellow waxes, hard fats, anhydrous dairy fats, lanolins, glyceryl palmitostearates, glyceryl stearates, lauric acid macrogolglycerides, cetyl alcohols, polyglyceryl diisostearates, diethylene glycol monostearates, ethylene monostearates, omegas 3 and mixtures of all or some of these waxes; and/or
from the group of fatty bases for suppositories comprising glycerol, triglycerides, theobroma oils, cacao butters and mixtures of all or some of these products.

6. The drug form as claimed in any one of the preceding claims, characterized in that it cannot be converted into a dry form that can be administered by nasal aspiration.

7. The drug form as claimed in any one of the preceding claims, characterized in that the viscosifying agent B) is chosen from those capable of rendering noninjectable the AI contained in the drug form.

8. The drug form as claimed in any one of the preceding claims, characterized in that the viscosifying agent B) is chosen from the following group of polymers:
polyacrylic acids and derivatives thereof, and/or
polyalkylene glycols (e.g. polyethylene glycol), and/or
polyvinylpyrrolidones, and/or
gelatins, and/or
polysaccharides, preferably from the subgroup comprising: sodium alginate, pectins, guars, xanthans, carrageenans, gellans and cellulose derivatives (e.g. hydroxypropylmethylcellulose, methylcellulose, hydroxyethylcellulose, carboxymethylcellulose),
and mixtures thereof.

9. The drug form as claimed in any one of the preceding claims, characterized in that it cannot be converted into an injectable form.

10. The drug form as claimed in claim 3, characterized in that the insoluble neutral beads are chosen from the group of following substances: celluloses and insoluble derivatives thereof, polymethacrylic resins and derivatives thereof, silicas, talc, semolina, bentonite and mixtures thereof.

11. The drug form as claimed in any one of the preceding claims, characterized in that it comprises immediate-release AI and/or modified-release AI.

12. The drug form as claimed in claims 3 and 11, characterized in that at least some of the microparticles are microcapsules for modified release of AI.

13. The drug form as claimed in claim 2 or 12, characterized in that the microparticles or the microcapsules have an average diameter of less than or equal to 1000 μm, preferably less than or equal to 500 μm, and more preferably a diameter of less than or equal to 300 μm.

14. The drug form as claimed in any one of the preceding claims, characterized in that the extraction of the AI by chewing and/or crushing is not effective.

15. The drug form as claimed in any one of the preceding claims, characterized in that the AI used belongs to at least one of the following families of active substances: amphetamines, analgesics, appetite suppressants, antidepressants, antiepileptics, antimigraine agents, antiparkinsonian agents, antitussives, anxiolytics, barbiturates, benzodiazepines, hypnotics, laxatives, neuroleptics, opiates, psychostimulants, psychotropic agents, sedatives and stimulants.

16. The drug form as claimed in one of the preceding claims, characterized in that the AI used is chosen from the following compounds: methylphenidate, Fentanyl, Alfentanyl, Pentazocine, Pethidine, Phenoperidine, Remifentanil, Sufentanil, Acetorphine, Acetylalphamethylfentanyl, Acetylmethadol, Alfentanil, Allylprodine, Alphacetylmethadol, Alphameprodine, Alphamethadol, Alphamethylfentanyl, Alpha-methylthiofentanyl, Alphaprodine, Anileridine, atropine, Benzethidine, Benzylmorphine, Beta-hydroxyfentanyl, Beta-hydroxymethyl-3-fentanyl, Betacetylmethadol, Betameprodine, Betamethadol, Betaprodine, Bezitramide, buprenorphine, Dioxaphentyl butyrate, Cannabis, Ketobemidone, Clonitazene, codeine, Coca, Cocaine, Codoxime, Concentrate of poppy straw, Desomorphine, Dextromoramide, Diampromide, Diethylthiambutene, Difenoxine, Dihydroetorphine, Dihydromorphine, Dimenoxadol, Dimepheptanol, Dimethylthiambutene, Diphenoxylate, Dipipanone, Drotebanol, Ecgonine, ephedrine, Ethylmethylthiambutene, Etonitazene, Etorphine, Etoxeridine, Fentanyl, Furethidine, Heroin, Hydrocodone, Hydromorphinol, Hydromorphone, Hydroxypethidine, . Isomethadone, Levomethorphane, Levomoramide, Levophenacylmorphane, Levorphanol, meperidine, Metazocine, Methadone, Methyldesorphine, Methyldihydromorphine, Methyl-3-thiofentanyl, Methyl-3-fentanyl, Metopon, Moramide, Morpheridine, morphine, MPPP, Myrophine, Nicomorphine, Noracymethadol, Norlevorphanol, Normethadone, Normorphine, Norpipanone, Opium, Oxycodone, Oxymorphone, Para-fluorofentanyl, PEPAP, Pethidine, Phenampromide, Phenazocine, Phenomorphane, Phenbperidine, Piminodine, Piritramide, Proheptazine, propanolol, Properidine, Racemethorphane, Racemoramide, Racemorphane, Remifentanil, Sufentanil, Thebacone, Thebaine, Thiofentanyl, Tilidine, Trimeperidine, Acetyldihydrocodeine, Codeine, Dextropropoxyphene, Dihydrocodeine, Ethylmorphine, Nicocodine, Nicodicodine, Norcodeine, Pholcodine, Propiram,
and mixtures thereof.
